(19) United States
(12) Patent Application Publication
(10) Pub. No.: US 2025/0249251 A1
(43) Pub. Date: Aug. 7, 2025

(54) SYSTEMS AND METHODS FOR STIMULATION OF THE VAGUS NERVE

(71) Applicant: Boston Scientific Neuromodulation Corporation, Valencia, CA (US)

(72) Inventors: Jianwen Gu, Richmond, TX (US); Rosana Esteller, Santa Clarita, CA (US); Ranjan Krishna Mukhari Nageri, Valencia, CA (US)

(21) Appl. No.: 19/043,984

(22) Filed: Feb. 3, 2025

Related U.S. Application Data
(60) Provisional application No. 63/549,783, filed on Feb. 5, 2024.

(51) Int. Cl. A61N 1/36; A61N 1/05

(52) U.S. Cl. CPC A61N 1/36053; A61N 1/0553; A61N 1/36139

(57) ABSTRACT

Methods and systems for stimulation of the vagus nerve of a patient using a stimulation system is described herein. The stimulation system including a control module and at least one stimulation lead coupleable to the control module, the at least one stimulation lead including a plurality of electrodes. The method includes implanting the at least one stimulation lead percutaneously or through an opening in the carotid sheath with placement of the electrodes proximate to the vagus nerve for stimulation of the vagus nerve; implanting the control module remote from the vagus nerve; generating stimulation signals using the control module; and delivering the stimulation signals from the control module through at least one of the electrodes of the at least one stimulation lead to stimulate the vagus nerve of the patient.

### CROSS-REFERENCE TO RELATED APPLICATIONS

This application claims the benefit under 35 U.S.C. § 119 (e) of U.S. Provisional Patent Application Ser. No. 63/549,783, filed Feb. 5, 2024, which is incorporated herein by reference.

### FIELD

The present disclosure is directed to methods and systems for stimulation of a patient. The present disclosure is also directed to methods and systems for stimulation of the vagus nerve of a patient.

### BACKGROUND

Implantable electrical stimulation systems have proven therapeutic in a variety of diseases and disorders. For example, deep brain stimulation systems have been used as a therapeutic modality for the treatment of Parkinson's disease, essential tremor, and the like.

Stimulators have been developed to provide therapy for a variety of treatments. A stimulator can include an implantable pulse generator (IPG), one or more leads, and an array of stimulator electrodes on each lead. The stimulator electrodes are in contact with or near the nerves, muscles, or other tissue to be stimulated. The pulse generator in the IPG generates electrical pulses that are delivered by the electrodes to body tissue.

### BRIEF SUMMARY

One aspect is a method for stimulation of the vagus nerve of a patient using a stimulation system, the stimulation system including a control module and at least one stimulation lead coupleable to the control module, the at least one stimulation lead including a plurality of electrodes. The method includes implanting the at least one stimulation lead percutaneously, laparoscopically, or through an incision of no more than 20 cm (or 15, 10, or 5 cm) with placement of the electrodes proximate to the vagus nerve for stimulation of the vagus nerve; implanting the control module remote from the vagus nerve; generating stimulation signals using the control module; and delivering the stimulation signals from the control module through at least one of the electrodes of the at least one stimulation lead to stimulate the vagus nerve of the patient.

Another aspect is a method for stimulation of the vagus nerve of a patient using a stimulation system, the stimulation system including a control module and at least one stimulation lead coupleable to the control module, the at least one stimulation lead including a plurality of electrodes. The method includes making an opening in a carotid sheath of the patient; implanting, through the opening, the at least one stimulation lead with placement of the electrodes proximate to the vagus nerve for stimulation of the vagus nerve, wherein the at least one stimulation lead does not entirely surround the vagus nerve; implanting the control module remote from the vagus nerve; generating stimulation signals using the control module; and delivering the stimulation signals from the control module through at least one of the electrodes of the at least one stimulation lead to stimulate the vagus nerve of the patient.

In at least some aspects, none of the at least one stimulation lead is a cuff lead or a lead that wraps around the vagus nerve. In at least some aspects, none of the at least one stimulation lead includes an electrode that surrounds or is wound around the vagus nerve.

In at least some aspects, the at least one stimulation lead includes a plurality of stimulation leads. In at least some aspects, the implanting includes implanting two of the stimulation leads on opposite sides of the vagus nerve. In at least some aspects, the implanting includes implanting at least three or four of the stimulation leads. In at least some aspects, the implanting includes implanting two of the stimulation leads axially offset from each other.

In at least some aspects, the at least one stimulation lead includes at least one paddle lead. In at least some aspects, the at least one stimulation lead includes a first stimulation lead including at least one set of segmented electrodes arranged around a perimeter of the first stimulation lead. In at least some aspects, the delivering includes delivering the stimulation signals using at least one of the segmented electrodes to target a particular portion of the vagus nerve. In at least some aspects, the delivering includes delivering the stimulation signals using at least two of the segmented electrodes, as either all cathodes or all anodes, to define a virtual cathode or virtual anode, respectively.

In at least some aspects, the delivering includes delivering the stimulation signals using at least one of the electrodes as a cathode and at least two of the electrodes as anodes flanking, both distally and proximally, the at least one of the electrodes used as a cathode. In at least some aspects, the delivering includes delivering the stimulation signal through the electrodes of the stimulation lead, wherein an anodic current delivered through the stimulation lead is larger in magnitude than a cathodic current delivered through the stimulation lead. In at least some aspects, the delivering includes delivering the stimulation signal using a housing of the control module as a cathode and at least one of the electrodes of the at least one stimulation lead as a cathode.

In at least some aspects, the method further includes delivering a test signal using at least one of the electrodes to identify a portion of the vagus nerve as a stimulation target. In at least some aspects, the method further includes obtaining an impedance measurement for each of a plurality of the electrodes, at least one combination of the electrodes, or any combination thereof. In at least some aspects, the method further includes determining a position of each of a plurality of the electrodes, relative to the nerve or to another of the electrodes, using the impedance measurements. In at least some aspects, the method further includes determining a tissue characteristic of tissue adjacent to each of a plurality of the electrodes using the impedance measurements. In at least some aspects, the method further includes selecting at least one of the electrodes for stimulation of the vagus nerve based, at least in part, on the impedance measurements.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified. For a better understanding of the present invention, reference will be made to the following Detailed Description, which is to be read in association with the accompanying drawings, wherein:

FIG. 1 is a schematic view of one embodiment of an electrical stimulation system that includes one or more leads that can be coupled to an IPG;

FIG. 2A is a schematic side view of one embodiment of an electrical stimulation system with a percutaneous or isodiametric (at least along a distal end portion) lead;

FIG. 2B is a schematic side view of one embodiment of an electrical stimulation system with a paddle lead;

FIG. 3A is a schematic side view of one embodiment of a control module receiving the proximal end portions of two leads or other elongated devices;

FIG. 3B is a schematic side view of one embodiment of a control module and lead extension receiving the proximal end portion of a lead or other elongated device;

FIG. 4 is a block diagram of elements of an electrical stimulation system;

FIG. 5 is a flowchart of one embodiment of a method for stimulation of the vagus nerve of a patient using a stimulation system; and

FIG. 6 is a schematic view of a distal end portion of stimulation lead implanted for stimulation of a vagus nerve of a patient;

FIG. 7 is a schematic view of distal end portions of two stimulation leads implanted for stimulation of a vagus nerve of a patient;

FIG. 8 is a schematic view of distal end portions of two axially offset stimulation leads implanted for stimulation of a vagus nerve of a patient; and

FIG. 9 is a schematic view of a distal end portion of paddle lead implanted for stimulation of a vagus nerve of a patient.

### DETAILED DESCRIPTION

The present disclosure is directed to methods and systems for stimulation of a patient. The present disclosure is also directed to methods and systems for stimulation of the vagus nerve of a patient.

Implantable electrical stimulation systems and devices are used herein to exemplify the inventions, but it will be understood that these inventions can be utilized with other stimulation or modulation systems and devices. Examples of implantable electrical stimulation systems include, but are not limited to, a least one lead with one or more electrodes disposed along a distal end of the lead and one or more terminals disposed along the one or more proximal ends of the lead. Examples of electrical stimulation systems with leads are found in, for example, U.S. Pat. Nos. 6,181,969; 6,295,944; 6,391,985; 6,516,227; 6,609,029; 6,609,032; 6,741,892; 7,244,150; 7,450,997; 7,672,734; 7,761,165; 7,783,359; 7,792,590; 7,809,446; 7,949,395; 7,974,706; 8,831,742; 8,688,235; 8,175,710; 8,224,450; 8,271,094; 8,295,944; 8,364,278; and 8,391,985; U.S. Patent Application Publications Nos. 2007/0150036;

2009/0187222; 2009/0276021; 2010/0076535; 2010/0268298; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; 2013/0105071; 2011/0005069; 2010/0268298; 2011/0130817; 2011/0130818; 2011/0078900; 2011/0238129; 2011/0313500; 2012/0016378; 2012/0046710; 2012/0165911; 2012/0197375; 2012/0203316; 2012/0203320; and 2012/0203321, all of which are incorporated by reference in their entireties.

Electrical stimulation systems are used in the description below but it will be understood that the electrical stimulation system, electrical stimulation lead, and electrodes can be replaced, in whole or in part, by an optical stimulation system (or electrical/optical stimulation system), optical stimulation lead, and optical emitters to produce optical stimulation or photobiomodulation. Examples of optical stimulation systems and electrical/optical stimulation systems, which include one or more optical emitters instead, or in addition to electrodes, are found in U.S. Pat. Nos. 9,415,154; 10,335,607; 10,625,072; and 10,814,140 and U.S. Patent Application Publications Nos. 2013/0317572; 2013/0317573; 2017/0259078; 2017/0225007; 2018/0110971; 2018/0369606; 2018/0369607; 2019/0209849; 2019/0209834; 2020/0094047; 2020/0155584; 2020/0376262; 2021/0008388; 2021/0008389; 2021/0016111; and 2022/0072329, all of which are incorporated by reference in their entireties.

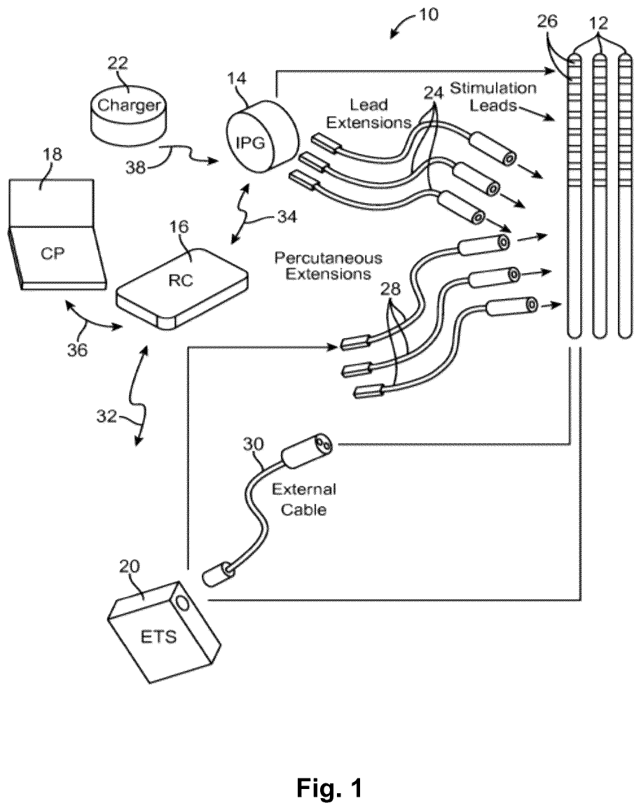

Turning to FIG. 1, one embodiment of an electrical stimulation system 10 includes one or more stimulation leads 12 and an implantable pulse generator (IPG) 14. The stimulation system 10 can also include one or more of an external remote control (RC) 16, a clinician's programmer (CP) 18, an external trial stimulator (ETS) 20, or an external charger 22. The IPG and ETS are examples of control modules for the electrical stimulation system.

The IPG 14 is physically connected, optionally via one or more lead extensions 24, to the stimulation lead(s) 12. Each lead carries multiple electrodes 26 arranged in an array. The IPG 14 includes pulse generation circuitry that delivers electrical stimulation energy in the form of, for example, a pulsed electrical waveform (i.e., a temporal series of electrical pulses) to the electrode array 26 in accordance with a set of stimulation parameter values. The implantable pulse generator can be implanted into a patient's body, for example, below the patient's clavicle area or within the patient's abdominal cavity or at any other suitable site. The implantable pulse generator 14 can have multiple stimulation channels which may be independently programmable to control the magnitude of the current stimulus from each channel. In some embodiments, the implantable pulse generator 14 can have any suitable number of stimulation channels including, but not limited to, 4, 6, 8, 12, 16, 32, or more stimulation channels. The implantable pulse generator 14 can have one, two, three, four, or more connector ports, for receiving the terminals of the leads and/or lead extensions.

The ETS 20 may also be physically connected, optionally via the percutaneous lead extensions 28 and external cable 30, to the stimulation leads 12. The ETS 20, which may have similar pulse generation circuitry as the IPG 14, also delivers electrical stimulation energy in the form of, for example, a pulsed electrical waveform to the electrode array 26 in accordance with a set of stimulation parameter values. One difference between the ETS 20 and the IPG 14 is that the ETS 20 is often a non-implantable device that is used on a trial basis after the neurostimulation leads 12 have been implanted and prior to implantation of the IPG 14, to test the responsiveness of the stimulation that is to be provided. Any functions described herein with respect to the IPG 14 can likewise be performed with respect to the ETS 20.

The RC 16 may be used to telemetrically communicate with or control the IPG 14 or ETS 20 via a uni- or bi-directional wireless communications link 32 or via any other wired or wireless communication technique. Once the IPG 14 and neurostimulation leads 12 are implanted, the RC 16 may be used to telemetrically communicate with or control the IPG 14 via a uni- or bi-directional communications link 34 or via any other wired or wireless communication technique. Such communication or control allows the IPG 14, for example, to be turned on or off and to be programmed with different stimulation parameter sets. The IPG 14 may also be operated to modify the programmed stimulation parameter values to actively control the characteristics of the electrical stimulation energy output by the IPG 14. In at least some embodiments, the CP 18 (or RC 16 or other programming device) allows a user, such as a clinician, the ability to program stimulation parameter values for the IPG 14 and ETS 20 in the operating room and in follow-up sessions. Alternately, or additionally, in at least some embodiments, stimulation parameter values can be programed via wireless communications (e.g., Bluetooth) between the RC 16 (or other external device such as a hand-held electronic device like a mobile phone, tablet, or the like) and the IPG 14.

The CP 18 may perform this function by indirectly communicating with the IPG 14 or ETS 20, through the RC 16, via a wireless communications link 36. Alternatively, the CP 18 may directly communicate with the IPG 14 or ETS 20 via a wireless communications link (not shown). In at least some embodiments, the stimulation parameter values provided by the CP 18 are also used to program the RC 16, so that the stimulation parameter values can be subsequently modified by operation of the RC 16 in a stand-alone mode (i.e., without the assistance of the CP 18). The CP 18 or RC 16 can be any suitable device including, but not limited to, a computer or other computing device, laptop, mobile device (for example, a mobile phone or tablet), or the like or any combination thereof. The CP 18 or RC 16 can include software applications for interacting with the IPG 14 or ETS 20 and for programming the IPG 14 or ETS 20.

Additional examples of the RC 16, CP 18, ETS 20, and external charger 22 can be found in the references cited herein as well as U.S. Pat. Nos. 6,895,280; 6,181,969; 6,516,227; 6,609,029; 6,609,032; 6,741,892; 7,949,395; 7,244,150; 7,672,734; and 7,761,165; 7,974,706; 8,175,710; 8,224,450; and 8,364,278; and U.S. Patent Application Publication No. 2007/0150036, all of which are incorporated herein by reference in their entireties.

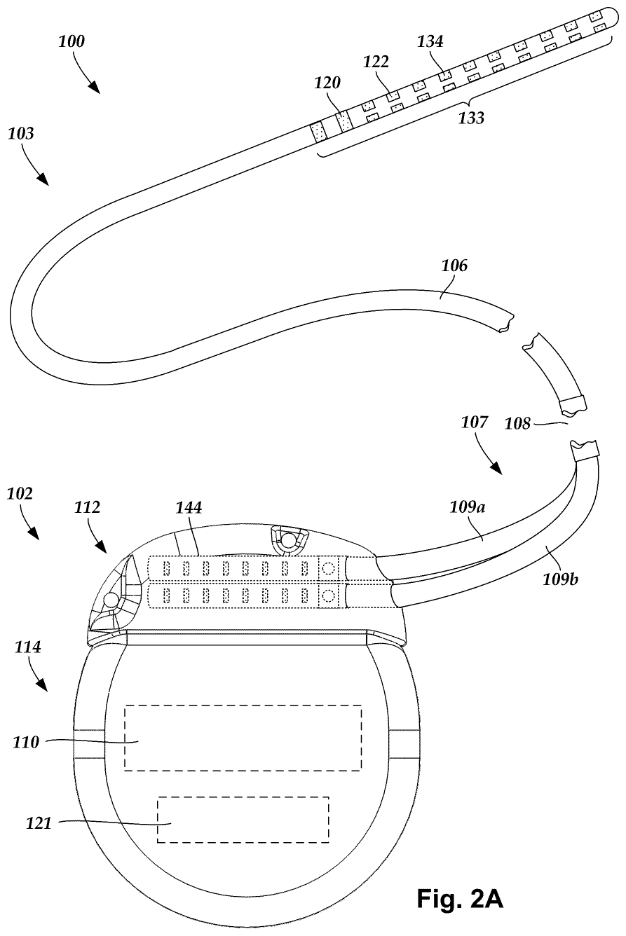

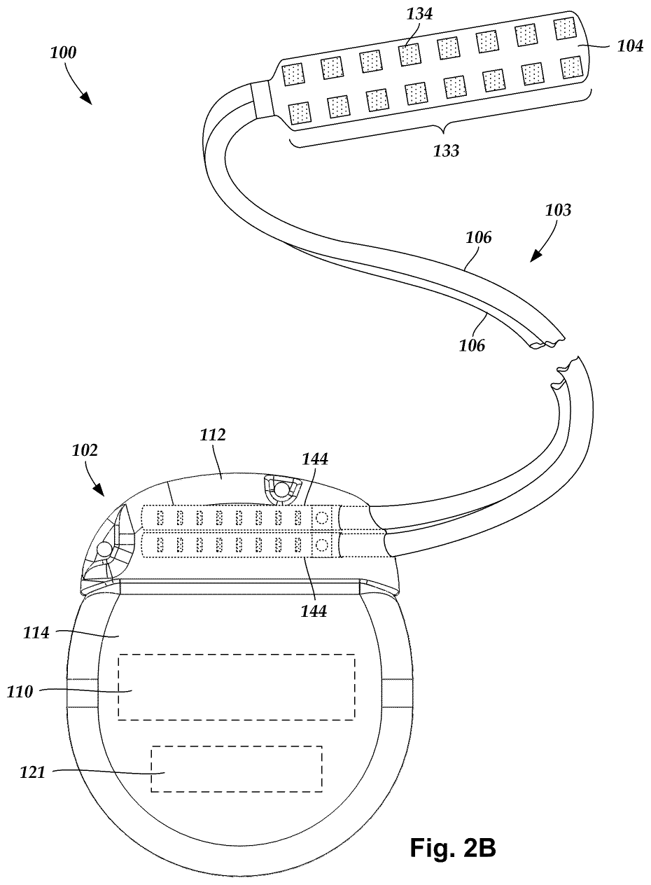

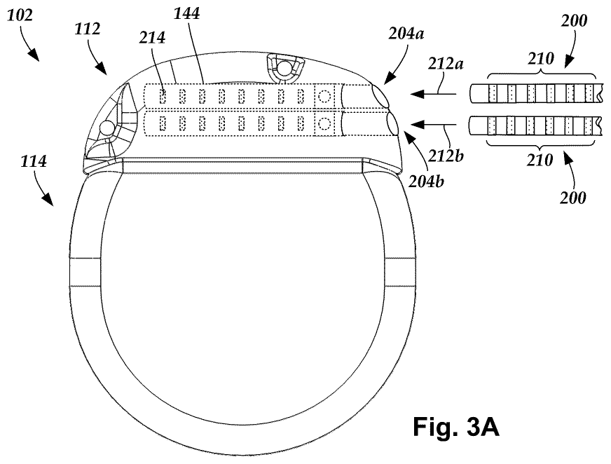

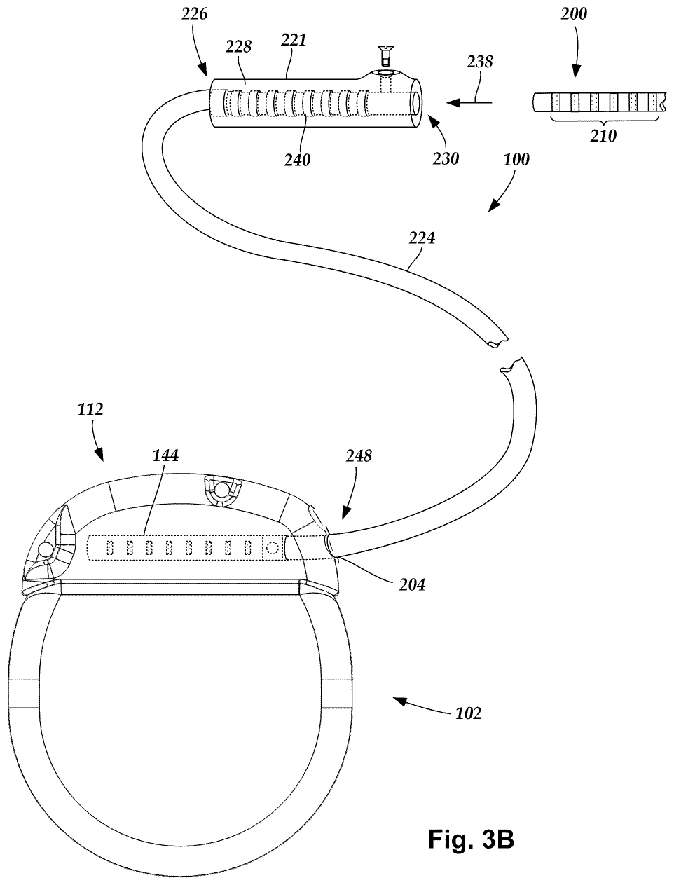

FIG. 2A illustrates schematically one embodiment of an electrical stimulation system 100. The electrical stimulation system includes a control module 102 (e.g., a stimulator or pulse generator, such as an implantable pulse generator (IPG 14) or an external test stimulator (ETS 20)) and at least one lead 103 coupleable to the control module 102. The lead 103 includes one or more lead bodies 106, an array of electrodes 133, such as electrode 134, and an array of terminals (e.g., 210 in FIGS. 3A and 3B) disposed along the one or more lead bodies 106. In at least some embodiments, the lead is isodiametric along a longitudinal length of the lead body 106. FIG. 2A illustrates one lead 103 coupled to a control module 102. Other embodiments may include two, three, four, or more leads 103 coupled to the control module 102.

The lead 103 can be coupled to the control module 102 in any suitable manner. In at least some embodiments, the lead 103 couples directly to the control module 102. In at least some other embodiments, the lead 103 couples to the control module 102 via one or more intermediate devices. For example, in at least some embodiments one or more lead extensions 224 (see e.g., FIG. 3B) can be disposed between the lead 103 and the control module 102 to extend the distance between the lead 103 and the control module 102. Other intermediate devices may be used in addition to, or in lieu of, one or more lead extensions including, for example, a splitter, an adaptor, or the like or combinations thereof. It will be understood that, in the case where the electrical stimulation system 100 includes multiple elongated devices disposed between the lead 103 and the control module 102, the intermediate devices may be configured into any suitable arrangement.

In FIG. 2A, the electrical stimulation system 100 is shown having an optional splitter 107 configured and arranged for facilitating coupling of the lead 103 to the control module 102. The splitter 107 includes a splitter connector 108 configured to couple to a proximal end of the lead 103, and one or more splitter tails 109a and 109b configured and arranged to couple to the control module 102 (or another splitter, a lead extension, an adaptor, or the like).

The control module 102 typically includes a connector housing 112 (e.g., a header) and a sealed electronics housing 114. An electronic subassembly 110 and an optional power source 121 are disposed in the electronics housing 114. In other embodiments, the control module 102 can receive power from another source including, but not limited to, an inductive power source disposed, for example, near or adjacent to the patient. A control module connector 144 is disposed in the connector housing 112. The control module connector 144 is configured and arranged to make an electrical connection between the lead 103 and the electronic subassembly 110 of the control module 102.

The electrical stimulation system or components of the electrical stimulation system, including one or more of the lead bodies 106 and the control module 102, are typically implanted into the body of a patient. The electrical stimulation system can be used for a variety of applications including, but not limited to, brain stimulation, neural stimulation, spinal cord stimulation, muscle stimulation, and the like.

The electrodes 134 can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of: platinum, platinum iridium, palladium, palladium rhodium, or titanium. The number of electrodes 134 in each array 133 may vary. For example, there can be two, four, six, eight, ten, twelve, fourteen, sixteen, or more electrodes 134. As will be recognized, other numbers of electrodes 134 may also be used.

The electrodes of the one or more lead bodies 106 are typically disposed in, or separated by, a non-conductive, biocompatible material such as, for example, silicone, polyurethane, polyetheretherketone (“PEEK”), epoxy, and the like or combinations thereof. The lead bodies 106 may be formed in the desired shape by any process including, for example, molding (including injection molding), casting, and the like. The non-conductive material typically extends from the distal end of the one or more lead bodies 106 to the proximal end of each of the one or more lead bodies 106.

In at least some embodiments, at least some of the stimulation electrodes take the form of segmented electrodes that extend only partially around the perimeter (for example, the circumference) of the lead. These segmented electrodes can be provided in sets of electrodes, with each set having electrodes circumferentially distributed about the lead at a particular longitudinal position.

In at least some embodiments, a practitioner may determine the position of the target neurons using recording electrode(s) and then position the stimulation electrode(s) accordingly. In some embodiments, the same electrodes can be used for both recording and stimulation. In some embodiments, separate leads can be used; one with recording electrodes which identify target neurons, and a second lead with stimulation electrodes that replaces the first after target neuron identification. In some embodiments, the same lead may include both recording electrodes and stimulation electrodes or electrodes may be used for both recording and stimulation.

In FIG. 2A, the electrodes 134 are shown as including both ring electrodes 120 and segmented electrodes 122. In some embodiments, the electrodes 134 are all segmented. The segmented electrodes 122 of FIG. 2A are in sets of three (one of which is not visible in FIG. 2A), where the three segmented electrodes of a particular set are electrically isolated from one another and are circumferentially distributed around the lead 103 at a particular longitudinal position along the lead. Any suitable number of segmented electrodes can be formed into a set including, for example, two, three, four, or more segmented electrodes. A lead can include any suitable number of sets of segmented electrodes including, for example, one, two, three, four, five, six, seven, eight, nine, ten, eleven, twelve, or more sets. The lead 103 of FIG. 2A has thirty segmented electrodes 122 (ten sets of three electrodes each) and two ring electrodes 120 for a total of 32 electrodes 134.

Segmented electrodes can be used to direct stimulus current to one side, or even a portion of one side, of the lead. When segmented electrodes are used in conjunction with an implantable pulse generator that delivers current stimulus, current steering can be achieved to more precisely deliver the stimulus to a position around an axis of the lead (i.e., radial positioning around the axis of the lead). Segmented electrodes may provide for superior current steering than ring electrodes because target structures in deep brain stimulation are not typically symmetric about the axis of the distal electrode array. Instead, a target may be located on one side of a plane running through the axis of the lead. Through the use of a segmented electrode array, current steering can be performed not only along a length of the lead but also around a perimeter of the lead. This provides precise three-dimensional targeting and delivery of the current stimulus to neural target tissue, while potentially avoiding stimulation of other tissue.

Examples of leads with segmented electrodes include U.S. Patent Application Publications Nos. 2010/0268298; 2011/0005069; 2011/0078900; 2011/0130803; 2011/0130816; 2011/0130817; 2011/0130818; 2011/0078900; 2011/0238129; 2011/0313500; 2012/0016378; 2012/0046710; 2012/0071949; 2012/0165911; 2012/0197375; 2012/0203316; 2012/0203320; 2012/0203321; 2013/0197602; 2013/0261684; 2013/0325091; 2013/0317587; 2014/0039587; 2014/0353001; 2014/0358209; 2014/0358210; 2015/0018915; 2015/0021817; 2015/0045864; 2015/0021817; 2015/0066120; 2013/0197424; 2015/0151113; 2014/0358207; and U.S. Pat. No. 8,483,237, all of which are incorporated herein by reference in their entireties. A lead may also include a tip electrode and examples of leads with tip electrodes include at least some of the previously cited references, as well as U.S. Patent Application Publications Nos. 2014/0296953 and 2014/0343647, all of which are incorporated herein by reference in their entireties. A lead with segmented electrodes may be a directional lead that can provide stimulation in a particular direction using the segmented electrodes.

Terminals (e.g., 210 in FIGS. 3A and 3B) are typically disposed along the proximal end of the one or more lead bodies 106 of the electrical stimulation system 100 (as well as any splitters, lead extensions, adaptors, or the like) for electrical connection to corresponding connector contacts (e.g., 214 in FIG. 3A and 240 in FIG. 3B). The connector contacts are disposed in connectors (e.g., 144 in FIGS. 2A, 2B, 3A, and 3B; and 221 in FIG. 3B) which, in turn, are disposed on, for example, the control module 102 (or a lead extension, a splitter, an adaptor, or the like). Electrically conductive wires, cables, or the like (not shown) extend from the terminals to the electrodes 134. Typically, one or more electrodes 134 are electrically coupled to each terminal. In at least some embodiments, each terminal is only connected to one electrode 134.

The electrically conductive wires (“conductors”) may be embedded in the non-conductive material of the lead body 106 or can be disposed in one or more lumens (not shown) extending along the lead body 106. In some embodiments, there is an individual lumen for each conductor. In other embodiments, two or more conductors extend through a lumen. There may also be one or more lumens (not shown) that open at, or near, the proximal end of the lead body 106, for example, for inserting a stylet to facilitate placement of the lead body 106 within a body of a patient. Additionally, there may be one or more lumens (not shown) that open at, or near, the distal end of the lead body 106, for example, for infusion of drugs or medication into the site of implantation of the one or more lead bodies 106. In at least one embodiment, the one or more lumens are flushed continually, or on a regular basis, with saline, epidural fluid, or the like. In at least some embodiments, the one or more lumens are permanently or removably sealable at the distal end.

FIG. 2B illustrates schematically another embodiment of an electrical stimulation system 100. The electrical stimulation system includes a control module (e.g., a stimulator or pulse generator) 102 and a lead 103 coupleable to the control module 102. The lead 103 includes a paddle body 104 and one or more lead bodies 106. In FIG. 2B, the lead 103 is shown having two lead bodies 106. It will be understood that the lead 103 can include any suitable number of lead bodies including, for example, one, two, three, four, five, six, seven, eight or more lead bodies 106. An array 133 of electrodes, such as electrode 134, is disposed on the paddle body 104, and an array of terminals (e.g., 210 in FIG. 3A-3B) is disposed along each of the one or more lead bodies 106.

FIG. 3A is a schematic side view of one embodiment of a proximal end of one or more elongated devices 200 configured and arranged for coupling to one embodiment of the control module connector 144. The one or more elongated devices may include, for example, the lead body 106, one or more intermediate devices (e.g., the splitter 107 of FIG. 2A, the lead extension 224 of FIG. 3B, an adaptor, or the like or combinations thereof), or a combination thereof. FIG. 3A illustrates two elongated devices 200 coupled to the control module 102. These two elongated devices 200 can be two tails as illustrated in FIGS. 2A and 2B or two different leads or any other combination of elongated devices.

The control module connector 144 defines at least one port 204a, 204b into which a proximal end of the elongated device 200 can be inserted, as shown by directional arrows 212a and 212b. In FIG. 3A (and in other figures), the connector housing 112 is shown having two ports 204a and 204b. The connector housing 112 can define any suitable number of ports including, for example, one, two, three, four, five, six, seven, eight, or more ports.

The control module connector 144 also includes a plurality of connector contacts, such as connector contact 214, disposed within each port 204a and 204b. When the elongated device 200 is inserted into the ports 204a and 204b, the connector contacts 214 can be aligned with a plurality of terminals 210 disposed along the proximal end(s) of the elongated device(s) 200 to electrically couple the control module 102 to the electrodes (134 of FIGS. 2A and 2B) disposed at a distal end of the lead 103. Examples of connectors in control modules are found in, for example, U.S. Pat. Nos. 7,244,150 and 8,224,450, which are incorporated herein by reference in their entireties.

FIG. 3B is a schematic side view of another embodiment of the electrical stimulation system 100. The electrical stimulation system 100 includes a lead extension 224 that is configured and arranged to couple one or more elongated devices 200 (e.g., the lead body 106, the splitter 107, an adaptor, another lead extension, or the like or combinations thereof) to the control module 102. In FIG. 3B, the lead extension 224 is shown coupled to a single port 204 defined in the control module connector 144. Additionally, the lead extension 224 is shown configured and arranged to couple to a single elongated device 200. In alternate embodiments, the lead extension 224 is configured and arranged to couple to multiple ports 204 defined in the control module connector 144, or to receive multiple elongated devices 200, or both.

A lead extension connector 221 is disposed on the lead extension 224. In FIG. 3B, the lead extension connector 221 is shown disposed at a distal end 226 of the lead extension 224. The lead extension connector 221 includes a connector housing 228. The connector housing 228 defines at least one port 230 into which terminals 210 of the elongated device 200 can be inserted, as shown by directional arrow 238. The connector housing 228 also includes a plurality of connector contacts, such as connector contact 240. When the elongated device 200 is inserted into the port 230, the connector contacts 240 disposed in the connector housing 228 can be aligned with the terminals 210 of the elongated device 200 to electrically couple the lead extension 224 to the electrodes (134 of FIGS. 2A and 2B) disposed along the lead (103 in FIGS. 2A and 2B).

In at least some embodiments, the proximal end of the lead extension 224 is similarly configured and arranged as a proximal end of the lead 103 (or other elongated device 200). The lead extension 224 may include a plurality of electrically conductive wires (not shown) that electrically couple the connector contacts 240 to a proximal end 248 of the lead extension 224 that is opposite to the distal end 226. In at least some embodiments, the conductive wires disposed in the lead extension 224 can be electrically coupled to a plurality of terminals (not shown) disposed along the proximal end 248 of the lead extension 224. In at least some embodiments, the proximal end 248 of the lead extension 224 is configured and arranged for insertion into a connector disposed in another lead extension (or another intermediate device). In other embodiments (and as shown in FIG. 3B), the proximal end 248 of the lead extension 224 is configured and arranged for insertion into the port 204 of the control module connector 144.

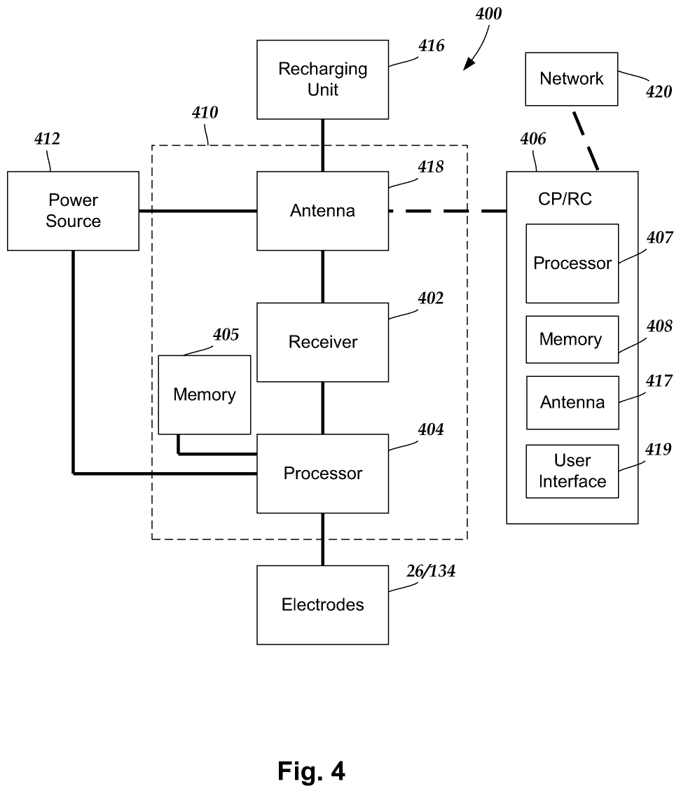

FIG. 4 is a schematic overview of one embodiment of components of an electrical stimulation system 400 including an electronic subassembly 410 disposed within an IPG 14 (FIG. 1). It will be understood that the electrical stimulation system can include more, fewer, or different components and can have a variety of different configurations including those configurations disclosed in the stimulator references cited herein.

The IPG 14 (FIG. 1) or control module 102 (FIGS. 2A to 3B) can include, for example, a power source 412, antenna 418, receiver 402, processor 404, and memory 405. Some of the components (for example, power source 412, antenna 418, receiver 402, processor 404, and memory 405) of the electrical stimulation system can be positioned on one or more circuit boards or similar carriers within a sealed housing of the IPG 14 (FIG. 1), if desired. Unless indicated otherwise, the term “processor” refers to both embodiments with a single processor and embodiments with multiple processors.

An external device, such as a CP or RC 406, can include a processor 407, memory 408, an antenna 417, and a user interface 419. The user interface 419 can include, but is not limited to, a display screen on which a digital user interface can be displayed and any suitable user input device, such as a keyboard, touchscreen, mouse, track ball, or the like or any combination thereof.

Any power source 412 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference in its entirety.

As another alternative, power can be supplied by an external power source through inductive coupling via the antenna 418 or a secondary antenna. The external power source can be in a device that is mounted on the skin of the user or in a unit that is provided near the user on a permanent or periodic basis.

If the power source 412 is a rechargeable battery, the battery may be recharged using the antenna 418, if desired. Power can be provided to the battery for recharging by inductively coupling the battery through the antenna to a recharging unit 416 external to the user. Examples of such arrangements can be found in the references identified above.

In one embodiment, electrical current is emitted by the electrodes 26/134 on the lead body to stimulate nerve fibers, muscle fibers, or other body tissues near the electrical stimulation system. A processor 404 is generally included to control the timing and electrical characteristics of the electrical stimulation system. For example, the processor 404 can, if desired, control one or more of the timing, frequency, amplitude, width, and waveform of the pulses. In addition, the processor 404 can select which electrodes can be used to provide stimulation, if desired. In some embodiments, the processor 404 may select which electrode(s) are cathodes and which electrode(s) are anodes. In some embodiments, the processor 404 may be used to identify which electrodes provide the most useful stimulation of the desired tissue. Instructions for the processor 404 can be stored on the memory 405. Instructions for the processor 407 can be stored on the memory 408.

Any processor 404 can be used for the IPG and can be as simple as an electronic device that, for example, produces pulses at a regular interval or the processor can be capable of receiving and interpreting instructions from the CP/RC 406 (such as CP 18 or RC 16 of FIG. 1) that, for example, allows modification of pulse characteristics. In the illustrated embodiment, the processor 404 is coupled to a receiver 402 which, in turn, is coupled to the antenna 418. This allows the processor 404 to receive instructions from an external source to, for example, direct the pulse characteristics and the selection of electrodes, if desired. Any suitable processor 407 can be used for the CP/RC 406.

Any suitable memory 405, 408 can be used including computer-readable storage media may include, but is not limited to, volatile, nonvolatile, non-transitory, removable, and non-removable media implemented in any method or technology for storage of information, such as computer readable instructions, data structures, program modules, or other data. Examples of computer-readable storage media include, but are not limited to, RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a processor.

In one embodiment, the antenna 418 is capable of receiving signals (e.g., RF signals) from an antenna 417 of a CP/RC 406 (see, CP 18 or RC 16 of FIG. 1) which is programmed or otherwise operated by a user. The signals sent to the processor 404 via the antenna 418 and receiver 402 can be used to modify or otherwise direct the operation of the electrical stimulation system. For example, the signals may be used to modify the pulses of the electrical stimulation system such as modifying one or more of pulse width, pulse frequency, pulse waveform, and pulse amplitude. The signals may also direct the electrical stimulation system 400 to cease operation, to start operation, to start signal acquisition, to stop signal acquisition, to start charging the battery, or to stop charging the battery. In other embodiments, the stimulation system does not include an antenna 418 or receiver 402 and the processor 404 operates as programmed.

Optionally, the electrical stimulation system 400 may include a transmitter (not shown) coupled to the processor 404 and the antenna 418 for transmitting signals back to the CP/RC 406 or another unit capable of receiving the signals. For example, the electrical stimulation system 400 may transmit signals indicating whether the electrical stimulation system 400 is operating properly or not or indicating when the battery needs to be charged or the level of charge remaining in the battery. The processor 404 may also be capable of transmitting information about the pulse characteristics so that a user or clinician can determine or verify the characteristics.

Transmission of signals can occur using any suitable method, technique, or platform including, but not limited to, inductive transmission, radiofrequency transmission, Bluetooth™, Wi-Fi, cellular transmission, near field transmission, infrared transmission, or the like or any combination thereof. In addition, the IPG 14 can be wirelessly coupled to the RC 16 or CP 18 using any suitable arrangement include direct transmission or transmission through a network, such as a local area network, wide area network, the Internet, or the like or any combination thereof. The CP 18 or RC 16 may also be capable of coupling to, and sending data or other information to, a network 420, such as a local area network, wide area network, the Internet, or the like or any combination thereof.

Electrical stimulation systems can be used for a variety of different purposes including, but not limited to, spinal cord stimulation, deep brain stimulation, and peripheral nerve stimulation. In particular, peripheral nerve stimulation includes the placement of electrodes to stimulate a particular nerve target. The vagus nerve is used herein as an example. It will be understood that the electrical stimulation systems and methods described herein can be used for the stimulation of other peripheral nerves.

Stimulation of the vagus nerve has been used to treat a variety of diseases, disorders, or conditions including, but not limited to, heart conditions, epilepsy, depression and other mood disorders, or the like. Many conventional vagus nerve stimulation systems include either a lead with wire electrodes spirally wrapped around the vagus nerve or a cuff lead with at least one cuff, with electrodes disposed thereon, wrapped around the vagus nerve. Both of these types of vagus nerve stimulation systems require invasive surgical procedures to open the carotid sheath of the patient, identify the vagus nerve, and wrap the spiral wire electrodes or the cuff(s) around the vagus nerve. In many instances, electrode or cuff placement around the vagus nerve is often cervical. In many instances, the electrode or cuff placement is on the left vagus nerve, which has less branching than the right vagus nerve.

This surgical procedure carries risks because, for example, the carotid sheath also includes the carotid artery and jugular vein. Nicking either the carotid artery or jugular vein can result in rapid blood loss. Moreover, the vagus nerve is not reliably found in the same place in the neck of different patients and another nerve, such as a facial nerve, can be mistaken for the vagus nerve. Incorrect stimulation of a facial or other non-vagus nerve can have deleterious effects. In addition, fat tissue in the neck may result in the vagus nerve being deeper within the neck than expected, requiring deeper incision. The variability in the position of the vagus nerve within the patient's neck can also increase the risk of nicking the carotid artery or jugular vein. In addition, wrapping the electrode or cuff around the vagus nerve can substantially hinder, or even prevent, removal of the electrode or cuff from the nerve when explantation is desired. For at least some embodiments of a stimulation system, it is desirable to place electrodes for stimulation using non-invasive implantation techniques or using techniques that do not include wrapping an electrode or cuff around the vagus nerve.

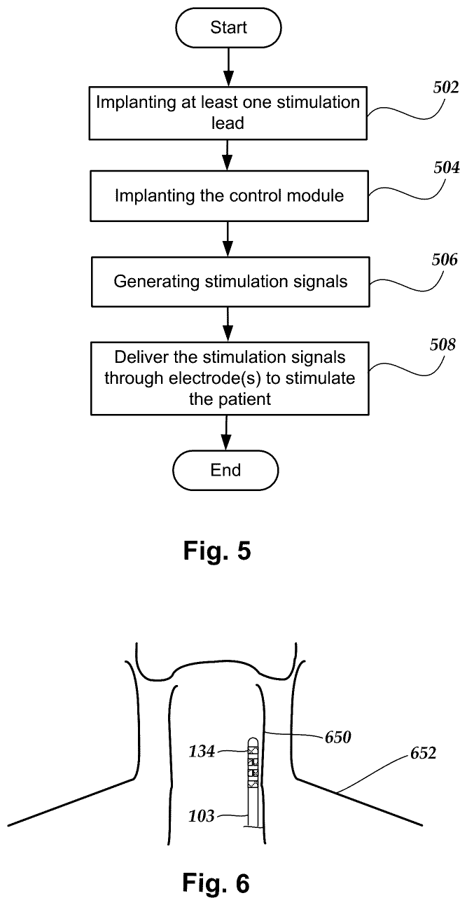

FIG. 5 is a flowchart of at least one embodiment of a method for stimulation of the vagus nerve using a stimulation system with at least one implantable stimulation lead having electrodes. This method can also be used for stimulation of other peripheral nerves. In step 502 at least one stimulation lead 103, 103a, 103b is implanted so that one or more (or all) of the electrodes 134 of the stimulation lead(s) are positioned proximate to the vagus nerve 650 of the patient 652 for stimulation of the vagus nerve (or other target nerve). Non-limiting examples of implantation of the stimulation lead(s) 103, 103a, 103b are illustrated in FIGS. 6-9 (where it will be understood that FIGS. 6-9 are not drawn to scale). One or more of the electrodes of each stimulation lead is implanted and positioned so that delivery of electrical stimulation through the electrode(s) can produce stimulation or modulation of the vagus (or other peripheral) nerve. In at least some embodiments, one or more (or all) of the electrodes 134 of each stimulation lead 103, 103a, 103b are positioned no more than 1, 2, 5, or 10 mm from the vagus nerve 650. Neither the lead(s) nor the electrodes are wrapped around the vagus nerve.

In at least some embodiments, one or more stimulation leads are implanted percutaneously, laparoscopically, or using a relatively small incision (for example, an incision of 20, 15, 10, or 5 mm or less) to position one or more electrodes proximate to the vagus nerve. For example, percutaneous or laparoscopic implantation can be performed using an insertion needle, lead introducer, cannula, or any other percutaneous or laparoscopic insertion tool through which the stimulation lead is delivered to the site of implantation. Non-limiting examples of insertion needles and lead introducers are found in, for example, U.S. Pat. Nos. 10,226,616 and 11,529,510 and U.S. Patent Application Publications Nos. 2011/0224680, 2014/0039586, 2014/0276927, 2015/0073431, 2015/0073432, 2016/0317800, and 2018/0333173, all of which are incorporated herein by reference in their entireties. The stimulation lead can be a cylindrical or isodiametric lead, a percutaneously-deliverable paddle lead, or any other suitable lead. In at least some embodiments, percutaneous or laparoscopic implantation can be assisted by imaging (e.g., fluoroscopy, MRI, PET, CAT, or camera imaging, or the like) before or during implantation or a combination thereof.

In at least some other embodiments, one or more stimulation leads can be implanted by making an incision into the neck of the patient to open the carotid sheath for placement of the stimulation lead(s) proximate the vagus nerve. The stimulation lead(s) can be inserted through the opening in the carotid sheath to position one or more of the electrodes of the stimulation lead(s) proximate to the vagus nerve. The stimulation lead can be a cylindrical or isodiametric lead, a paddle lead, or any other suitable lead. This implantation method does not include wrapping any electrode or other part of the stimulation lead(s) around the nerve.

Any other suitable implantation procedure, including any other suitable noninvasive implantation procedure, can be used to implant the stimulation lead(s) to place electrodes proximate to the vagus nerve. These implantation methods do not include wrapping any electrode or other part of the stimulation lead(s) around the nerve.

In at least some embodiments using any suitable implantation technique, the implanted stimulation lead(s) can be stabilized or secured in the implanted position using sutures, at least one lead anchor, a suture sleeve, anchoring units on the stimulation lead(s), or the like or any combination thereof. Non-limiting examples of devices and methods for stabilizing or securing a stimulation lead to tissue can be found at U.S. Pat. Nos. 8,019,443; 8,359,107; 8,412,349; 8,467,883; 8,751,016; 8,936,622; 8,986,382; 9,039,740; 9,533,141; 9,610,435; 9,636,498; 9,649,489; 9,669,210; 9,887,470; 9,987,482; 10,071,242; 10,406,353; 10,709,886; 10,835,739; and 10,857,351; and U.S. Patent Applications Publication Nos. 2014/0081366 and 2019/0105503, all of which are incorporated herein by reference in their entireties. In at least some embodiments, the stimulation lead(s) can be secured to the carotid sheath. In at least some embodiments, a stimulation lead 103, 103a, 103b has an extended distal portion (as compared to, for example, many stimulation leads for deep brain stimulation) or a suture eyelet or the like (or any combination thereof) distal to all of the electrodes that facilitates securing the distal end portion of the stimulation lead to the tissue (for example, the carotid sheath) of the patient.

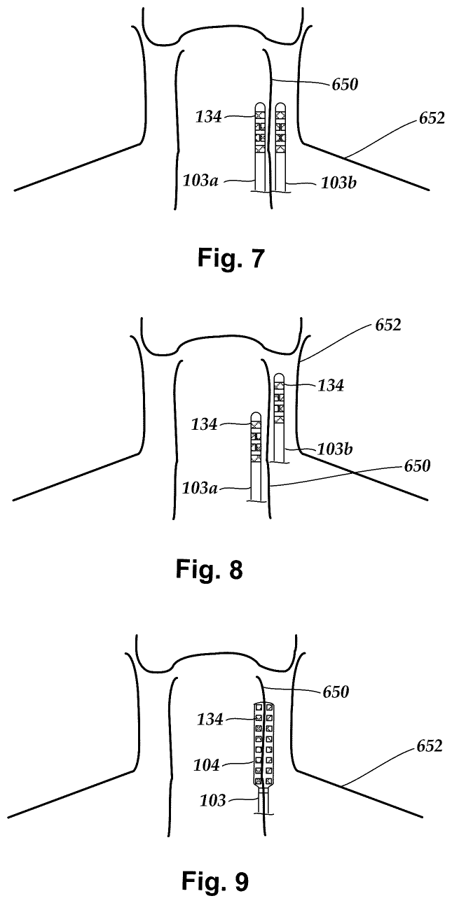

In at least some embodiments, two, three, four, or more stimulation leads 103, 103a, 103b are implanted. The stimulation leads can be the same (e.g., the same size, number of electrodes, electrode arrangement, or the like) or different (e.g., different size, different number of electrodes, different electrode arrangement, or any combination thereof). In at least some embodiments, two of the stimulation leads 103a, 103b are implanted on opposite sides (e.g., with separation between the stimulation leads of 150 to 210 degree when using the positions of the stimulation leads to define a circle around the nerve) of the vagus nerve 650, as illustrated in FIGS. 7 and 8. In FIGS. 7 and 8, the two stimulation leads 103a, 103b are disposed to the right and left of the vagus nerve. It will be understood that any other suitable opposing positions can be used including, but not limited to, anterior and posterior to the vagus nerve. Any additional stimulation leads can be implanted at other circumferential positions around the vagus lead. For example, three stimulation leads can be implanted around the vagus nerve (e.g., at approximately 120 degree increments or at increments of approximately 90, 90, and 180 degrees or any other suitable arrangement) or four stimulation lead can be implanted around the vagus nerve (e.g., at approximately 90 degree increments or any other suitable arrangement). In at least some embodiments, the term “approximately” in the previous sentence can mean within 5, 10, or 15 degrees.

In at least some embodiments, two of the implanted stimulation leads 103a, 103b are positioned with the electrodes 134 axially (i.e., longitudinally, in a direction defined by the long axis of the stimulation lead or of the portion of the stimulation lead containing the electrodes) aligned with each other, as illustrated in FIG. 7. In at least some embodiments, two of the implanted stimulation leads 103a, 103b are positioned with the electrodes 134 axially offset from each other, as illustrated in FIG. 8. In at least some embodiments, axially offset stimulation leads can axially extend the possible stimulation field that can be produced by the stimulation leads and axially extend the length of the vagus nerve that can be stimulated.

The stimulation lead(s) can include ring electrodes, segmented electrodes, or any combination thereof. In at least some embodiments, segmented electrodes can be used to direct a larger part of the stimulation field toward the vagus nerve as compared to ring electrodes. This can increase the stimulation efficiency of the stimulation system. In at least some embodiments, segmented electrodes can be used to facilitate targeting a particular fiber or group of fibers of the vagus nerve.

In at least some embodiments, none of the least one implanted stimulation lead is a cuff lead or a lead that wraps around the vagus nerve. In at least some embodiments, none of the at least one implanted stimulation lead includes an electrode that surrounds or is wound around the vagus nerve.

FIG. 9 illustrates the implantation of a stimulation lead 103 with a paddle body 104. In the illustrated embodiments, the paddle body 104 is implanted posterior to the vagus nerve 650, but it will be understood that the paddle body can be implanted medially, laterally, anteriorly, or at any other position relative to the vagus nerve. The illustrated paddle body 104 has two columns of electrodes 134. It will be understood that a paddle body 104 can have any suitable number of columns of electrodes 134 including, but not limited to, one, two, three, four, or more columns.

A stimulation lead 103, 103a, 103b can have any suitable number of electrodes 134 including, but not limited to, two, four, eight, twelve, sixteen, or more electrodes. For example, in at least some embodiments, each lead 103, 103a, 103b in FIGS. 6-8 has four, eight, twelve, or sixteen or more electrodes or the paddle lead 103 in FIG. 9 has eight, twelve, sixteen, or more electrodes. Any other suitable number of electrodes can be used on the leads 103, 103a, 103b in FIGS. 6-9.

In step 504, the control module is implanted. In at least some embodiments, the control module is implanted remote from the vagus nerve. For example, the control module can be implanted in the pectoral region or elsewhere in the torso including any of the sites used for the control modules of spinal cord or deep brain stimulation systems. The stimulation lead(s) can be tunneled to the remote control module.

The stimulation lead(s) can be permanently or removably attached to the control module. Attachment of the leads to the control module can occur before or after implantation of the stimulation lead(s) or before or after implantation of the control module.

In at least some embodiments, the control module includes a battery, for example, a rechargeable battery. In at least some embodiments, the control module is powered using an external power source by inductive coupling, radiofrequency transmission, or ultrasonic transmission. It will be understood that an electrical stimulation system can include multiple control modules with one or more stimulation leads coupled to each of the control modules.

In step 506, stimulation signals are generated using the control module. In step 508, the stimulation signals are delivered through at least one of the electrodes of the stimulation lead(s) to stimulate the vagus nerve of the patient. Examples of the generation and delivery of stimulation signals using a control module and stimulation lead(s) are found in the references cited above.

In at least some embodiments, a test signal can be directed through at least one of the electrodes to identify a portion of the vagus nerve that can be stimulated using the electrode(s). In at least some embodiments, the effect of the test signal can be determine using sensors (e.g., sensors of electrical signals of the nerve (for example, evoked resonant neural activity (ERNA) or evoked compound action potential (ECAP) or the like) or sensors that detect patient symptoms or side effects (e.g., tremor, rigidity, heart or brain signals, or the like)), patient feedback, clinician or programmer observation, or the like or any combination thereof. In at least some embodiments, the testing of one or more electrodes or one or more combinations of electrodes or any combination thereof can be used to identify portions of the vagus nerve (e.g., fibers or groups of fibers) that can be stimulated using the implanted stimulation lead(s).

In at least some embodiments, the selection of electrodes as cathode(s) and anode(s) can be used to target a particular portion of the vagus nerve, for example, a particular fiber or group of fibers of the vagus nerve, or the like. Electrodes can be selected from the same or different stimulation lead(s). For example, a cathode can be selected from one stimulation lead and an anode from another stimulation lead. Segmented electrodes on the stimulation lead(s) can also be used to enhance the directionality of the stimulation. For example, a ring of three segmented electrodes can be programmed to have one cathode and two anodes, where the cathode is closest to the vagus nerve and the anodes are further away. The anodes push the stimulation field towards the vagus nerve by acting as guarding anodes.

In at least some embodiments, multiple electrodes can be selected as cathodes and multiple electrodes can be selected as anodes. The references cited above include examples of stimulation systems that include this capability including examples of stimulation systems using multiple signal sources, a division of a signal source into multiple signals, switches, a multiplexor, or the like or any combination thereof.

Using multiple electrodes as a cathode or an anode can be characterized as a virtual electrode (e.g., a virtual cathode or a virtual anode) between the selected physical electrodes. In at least some embodiments, his arrangement can facilitate more precise directionality to the stimulation. Fractionalization refers to the distribution of the stimulation amplitude among the selected electrodes. For example, labelling the electrodes from distal to proximal with numbers 1, 2, . . . , if electrodes 2 and 4 are selected as cathodes and electrode 3 is selected as an anode, the cathodic stimulation amplitude could be distributed in a number of different ways (i.e., fractionalizations) among electrodes 2 and 4. Examples include, but are not limited to, 50% of the cathodic stimulation amplitude on each electrode, 67% on one electrode and 33% on the other electrode, 75% on one electrode and 25% on the other electrode, and so on. Different fractionalizations and electrode selections can be represented by different virtual electrodes and produce different stimulation fields. Such selections can facilitate directing the stimulation toward a desired target portion of the vagus nerve (for example, a target fiber or group of fibers).

In at least some embodiments, to enhance directionality or target selectivity, anode (or cathode) intensification or guarding can be used. In at least some embodiments, anode guarding can be achieved by selecting electrodes for anodes flanking the electrode(s) selected for cathode(s).

In at least some embodiments, anode intensification can be achieved by assigning a substantial portion (for example, at least 10, 15, 25, 30, 33, 35, or 40 percent) of the cathodic current to the housing or case of the control module (or to another remote electrode, such as an electrode on a stimulation lead that is more distant from the other cathode(s) than the anode(s)) so that the anode(s) appear to be relatively stronger than the cathode(s) delivering the remainder of the cathodic current. As an example, a portion of the cathodic current (for example, at least 40, 50, 60, 70, 80, or 90% of the total cathodic current) is assigned to one or more electrodes of the stimulation lead(s) and the remainder of the cathodic current is assigned to the housing of the control module (or another remote electrode). The anodic current can be assigned to electrodes flanking the electrodes assigned the cathodic current. This can result in anode intensification of the cathodic current. In at least some embodiments, anode intensification is used for tripolar stimulation. In at least some embodiments, the anodic electrodes can be used to reduce the leakage of current beyond the cathodic electrode(s) by creating a sink or source with opposite polarity to the cathodic electrodes. It will be understood that cathode guarding or intensification can be similarly produced by switching the polarities of the electrodes in the preceding description of anodic guarding or intensification.

In at least some embodiments, unidirectional propagating action potentials can be generated using one or more cathodes. In at least some embodiments, a strong anode can block action potentials in the non-propagating direction. It will be understood that a similar effect can be produced by switching the polarities of the electrodes.

In at least some embodiments, impedance measurements can be made for one or more of the electrodes or one or more combinations of electrodes or any combination thereof. In at least some embodiments, the impedance measurement(s) can be used to estimate or determine a position of one or more of the electrodes relative to the nerve or to other electrodes. In at least some embodiments, the impedance measurement(s) can be used to measure, determine, or estimate one or more tissue characteristics of tissue adjacent to the electrode(s) or combination(s) of electrodes. In at least some embodiments, the impedance measurements can be used to select, or as guidance for the selection of, at least one of the electrodes for stimulation of the vagus nerve.

It will be understood that each block of the flowchart illustration, and combinations of blocks in the flowchart illustration and methods disclosed herein, can be implemented by computer program instructions. These program instructions may be provided to a processor to produce a machine or engine, such that the instructions, which execute on the processor, create means for implementing the actions specified in the flowchart block or blocks or engine disclosed herein. The computer program instructions may be executed by a processor to cause a series of operational steps to be performed by the processor to produce a computer implemented process. The computer program instructions may also cause at least some of the operational steps to be performed in parallel. Moreover, some of the steps may also be performed across more than one processor, such as might arise in a multi-processor computing device. In addition, one or more processes may also be performed concurrently with other processes, or even in a different sequence than illustrated without departing from the scope or spirit of the invention.

The computer program instructions can be stored on any suitable computer-readable medium including, but not limited to, RAM, ROM, EEPROM, flash memory or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device. The computer program instructions can be stored locally or nonlocally (for example, in the Cloud).

The above specification and examples provide a description of the arrangement and use of the invention. Since many embodiments of the invention can be made without departing from the spirit and scope of the invention, the invention also resides in the claims hereinafter appended.

What is claimed as new and desired to be protected is:

1. A method for stimulation of the vagus nerve of a patient using a stimulation system, the stimulation system comprising a control module and at least one stimulation lead coupleable to the control module, the at least one stimulation lead comprising a plurality of electrodes, the method comprising:
making an opening in a carotid sheath of the patient;
implanting, through the opening, the at least one stimulation lead with placement of the electrodes proximate to the vagus nerve for stimulation of the vagus nerve, wherein the at least one stimulation lead does not entirely surround the vagus nerve;
implanting the control module remote from the vagus nerve;
generating stimulation signals using the control module; and
delivering the stimulation signals from the control module through at least one of the electrodes of the at least one stimulation lead to stimulate the vagus nerve of the patient.

2. The method of claim 1, wherein the at least one stimulation lead comprises a plurality of stimulation leads.

3. The method of claim 2, wherein the implanting comprises implanting two of the stimulation leads on opposite sides of the vagus nerve.

4. The method of claim 2, wherein the implanting comprises implanting at least three or four of the stimulation leads.

5. The method of claim 2, wherein the implanting comprises implanting two of the stimulation leads axially offset from each other.

6. The method of claim 1, wherein the at least one stimulation lead comprises at least one paddle lead.

7. The method of claim 1, wherein the at least one stimulation lead comprises a first stimulation lead comprising at least one set of segmented electrodes arranged around a perimeter of the first stimulation lead.

8. The method of claim 7, wherein the delivering comprises delivering the stimulation signals using at least one of the segmented electrodes to target a particular portion of the vagus nerve.

9. The method of claim 7, wherein the delivering comprises delivering the stimulation signals using at least two of the segmented electrodes, as either all cathodes or all anodes, to define a virtual cathode or virtual anode, respectively.

10. The method of claim 1, wherein the delivering comprises delivering the stimulation signals using at least one of the electrodes as a cathode and at least two of the electrodes as anodes flanking, both distally and proximally, the at least one of the electrodes used as a cathode.

11. The method of claim 10, wherein the delivering comprises delivering the stimulation signal through the electrodes of the stimulation lead, wherein a anodic current delivered through the stimulation lead is larger in magnitude than an cathodic current delivered through the stimulation lead.

12. The method of claim 10, wherein the delivering comprises delivering the stimulation signal using a housing of the control module as a cathode and at least one of the electrodes of the at least one stimulation lead as a cathode.

13. The method of claim 1, further comprising delivering a test signal using at least one of the electrodes to identify a portion of the vagus nerve as a stimulation target.

14. The method of claim 1, further comprising obtaining an impedance measurement for each of a plurality of the electrodes, at least one combination of the electrodes, or any combination thereof.

15. The method of claim 14, further comprising determining a position of each of a plurality of the electrodes, relative to the nerve or to another of the electrodes, using the impedance measurements.

16. The method of claim 14, further comprising determining a tissue characteristic of tissue adjacent to each of a plurality of the electrodes using the impedance measurements.

17. The method of claim 14, further comprising selecting at least one of the electrodes for stimulation of the vagus nerve based, at least in part, on the impedance measurements.

18. A method for stimulation of the vagus nerve of a patient using a stimulation system, the stimulation system comprising a control module and at least one stimulation lead coupleable to the control module, the at least one stimulation lead comprising a plurality of electrodes, the method comprising:
implanting the at least one stimulation lead percutaneously with placement of the electrodes proximate to the vagus nerve for stimulation of the vagus nerve;
implanting the control module remote from the vagus nerve;
generating stimulation signals using the control module; and
delivering the stimulation signals from the control module through at least one of the electrodes of the at least one stimulation lead to stimulate the vagus nerve of the patient.

19. The method of claim 18, wherein the at least one stimulation lead comprises a plurality of stimulation leads and the implanting comprises implanting two of the stimulation leads on opposite sides of the vagus nerve.

20. The method of claim 18, wherein the at least one stimulation lead comprises a plurality of stimulation leads and the implanting comprises implanting two of the stimulation leads axially offset from each other.
